(19) United States
(12) Patent Application Publication
(10) Pub. No.: US 2005/0056288 A1
(43) Pub. Date: Mar. 17, 2005

(54) Earplug and a method of forming an earplug

(76) Inventor: Walter Schreiber, Bear, DE (US)

Correspondence Address: CANTOR COLBURN, LLP, 55 GRIFFIN ROAD SOUTH, BLOOMFIELD, CT, 06002, US

(21) Appl. No.: 10/660,015

(22) Filed: Sep. 11, 2003

(51) Int. Cl. A61F 11/00

(52) U.S. Cl. 128/864; 128/857

(57) ABSTRACT

An earplug and a method of forming an earplug are provided, the method including providing a sheet of a compressible, resilient material, positioning the sheet proximate a water jet assembly, activating the water jet assembly to emit a high pressure water stream, and contacting the sheet with the water stream cutting the sheet and severing the earplug from the sheet.

### BACKGROUND OF INVENTION

(a) Field of Invention

The invention relates generally to hearing protection devices and, more particularly to a method of forming an earplug.

(b) Description of Related Art

The use of hearing protective and noise attenuating devices is well known, and various types of devices are available including, but not limited to, ear muffs, semi-aural devices, and earplugs. Earplugs are often preferred for their effectiveness in attenuating sound and for comfort properties provided thereby.

An earplug generally comprises a sound attenuating element which is placed in the ear canal of a wearer to provide a desired sound attenuation. The sound attenuating element is commonly made of a resiliently compressible, full recovery material such as a foam or a rubber. Particularly, such sound attenuating elements are often formed of a thermoplastic elastomer.

The earplug may further include a semi-rigid stem or a core embedded partly or entirely in the resilient sound attenuating element. The stem or core provides a degree of rigidity to the earplug which enables the earplug to be easily inserted and pushed into the ear canal of a user. Alternatively, an earplug may not include such a stem or core and, instead, is comprised primarily of the resilient sound attenuating element which is rolled between the fingers or hands to narrow a diameter thereof in order to facilitate insertion of the plug into the ear canal.

Resilient sound attenuating elements for earplugs are typically formed by conventional methods which employ molding, extrusion, and die cutting techniques.

In such molding processes, a mold is provided to shape the sound attenuating element. The resilient material, in liquid form, is injected into the mold and allowed to set therein. Once the material is solidified, the sound attenuating element is ejected from the mold.

Such molding techniques, however, are often not sufficiently efficient. For example, if a manufacturer desires several differently shaped earplugs, he or she must produce and maintain an equivalently corresponding number of molds. Further, the material used to form the sound attenuating element may stick to the mold during the solidification process and thus tear or otherwise deform upon ejection. Also, sound attenuating elements cast in a mold as described may include seam lines from the mold and also necessarily include any imperfections on the molding surface of the mold.

Extrusion formation of earplug sound attenuating elements involves the resilient material being formed within and released from an extruder as an extrudate, typically in a form of an elongated rod shape. The extrudate is often cylindrical and of a diameter just slightly larger than a typical ear canal. Once the rod-shaped resilient material is formed and extruded, it is trans-axially cut repeatedly to form a plurality of sound attenuating elements. That is, the extruded rod is severed perpendicularly to its longitudinal axis, for example, every 17-25 mm to form individual sound attenuating elements.

However, such extrusion techniques are often found to be less than desirable. Precise formation of the rod-shaped resilient material during extrusion is sometimes difficult to control. The resilient rod may over or under expand, radially, during extrusion, thus resulting in an inconsistently dimensioned extrudate. Also, the rod may inherit various imperfections of the extruder nozzle while being forced therethrough which then necessarily flaw the resulting sound attenuating elements. Additionally, cutting the extruded resilient rod into individual sound attenuating elements often proves difficult. An attempt at severing the rod can result in tearing of the resilient material thus flawing the extrudate. Alternatively, during the cutting, the rod may undesirably compress before severing. This compression, if occurring during a particular stage of formation of the resilient material, may be permanent thus resulting in pinched ends of the produced sound attenuating element. Also, some cutting techniques may evolve heat which can further degrade the extruded rod and hence the resulting sound attenuating elements.

Die cut formation of earplug sound attenuating elements involves production of a sheet of the resilient material and then punch-cutting individual sound attenuating elements from the sheet with a cutting die. For example, where a cylindrical element is desired, the die has a corresponding cylindrical shape such that when the die is pressed into the sheet of resilient material, a cylindrical portion is separated therefrom.

Die cutting, as with the previously discussed methods of sound attenuating element production, has its deficiencies. For example, the severing obtained from die cutting may be somewhat crude in nature. That is, cut surfaces of the resilient material may include various imperfections such as small protrusions, cavities, tears, etc. Further, the sheet of resilient material may be permanently compressed during die cutting. That is, prior to severing the sound attenuating element, the die may pinch and permanently deform the material. Additionally, due to the nature of die cutting, the shape of cut sound attenuating elements is limited to, at best, basic cylinders or polygons. Finally, die cutting of sound attenuating elements results in significant wasted material because the cutting precision of the die is extremely limited.

It is often desired to produce an earplug resilient sound attenuating element which includes an angled or sloped shape, an ornamental design etched or otherwise provided thereon, or a cavity formed therethrough. Such particular details are typically not capable of being readily and consistently formed by the above-discussed conventional manufacturing methods.

For example, where the sound attenuating element is formed in the molding process, the mold may include features which form the mentioned items and/or shapes, in situ, during molding. However, such a molding technique often results in inconsistent formation (i.e., inconsistent dimensioning and placement) of the described items.

Items such as inset ornamentation, angled or sloped shaping, and holes may be difficult to form via conventional extrusion or die casting production methods. Particularly, such features may be formed inconsistently or require subsequent processing steps to complete.

Thus, a method of consistently and efficiently forming an earplug sound attenuating element which provides the necessary precision to shape and ornament the element as desired, is needed.

### SUMMARY OF INVENTION

The above discussed and other problems and deficiencies of the prior art are overcome or alleviated by the hearing protective device and method of manufacture of the invention.

The invention provides a method of forming an earplug including providing a sheet of a compressible, resilient material, positioning the sheet proximate a water jet assembly, activating the water jet assembly to emit a high pressure water stream, and contacting the sheet with the water stream cutting the sheet and severing the earplug from the sheet.

The above discussed and other features and advantages of the present invention will be appreciated and understood by those skilled in the art from the following detailed description and drawings.

### BRIEF DESCRIPTION OF DRAWINGS

Referring now to the drawings wherein like elements are numbered alike in the several FIGURES:

FIG. 1 is an elevational view of an earplug according to one embodiment of the invention;

FIG. 2 is a cross-sectional view thereof taken along the line I-I of FIG. 1;

FIG. 3 is perspective view of an earplug in another embodiment of the invention;

FIG. 4 is a cross-sectional view thereof taken along line II-II of FIG. 3 with a hole shown in one embodiment;

FIG. 5 is a cross-sectional view thereof taken along line II-II of FIG. 3 with the hole shown in another embodiment and including an insert;

FIG. 6 is a cross-sectional view thereof taken along line II-II of FIG. 3 with the hole shown in another embodiment and including an insert;

FIG. 7 is a elevational view of an earplug in another embodiment of the invention;

FIG. 8 is a elevational view of an earplug in another embodiment of the invention;

FIGS. 9A-9B are elevational views of earplugs in another embodiment of the invention; and

FIG. 10 is a schematic representation of a method of manufacturing the earplug of the invention including a water jet assembly.

### DETAILED DESCRIPTION OF INVENTION

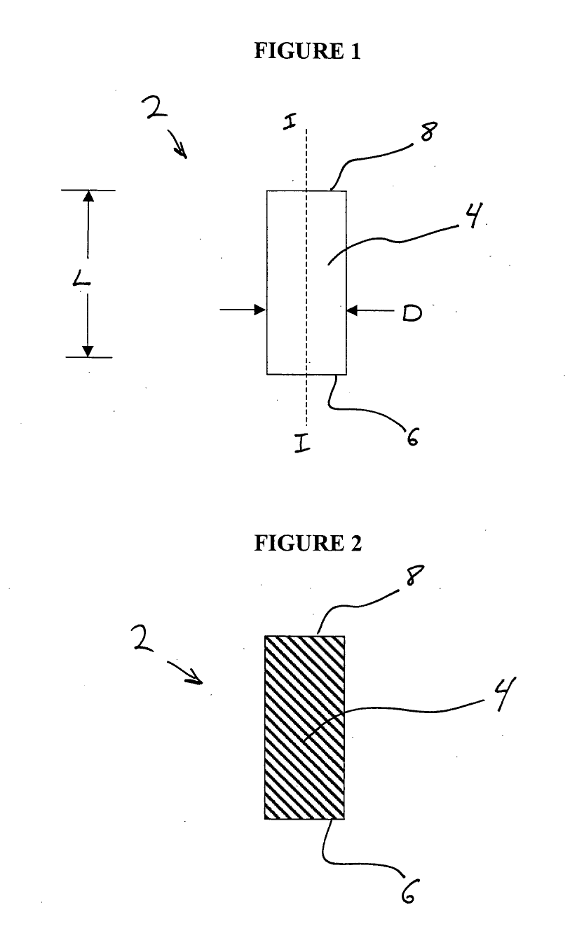

FIG. 1 shows an earplug 2 according to one embodiment of the invention. The earplug 2 includes a sound attenuating element 4 generally composed of a compressible, resilient full-recovery material such as, for example, a foam or rubber material. In one embodiment, the sound attenuating element 4 is composed of an elastomer. The sound attenuating element 4 includes a first end 6 and an opposite second end 8. The sound attenuating element 4 is generally cylindrical in shape and includes length L and a diameter D which is slightly larger than a diameter of an ear canal of the user. The sound attenuating element, in this embodiment, is formed monolithically of the compressible, resilient material. FIG. 2 shows a cross-section of the earplug 2 taken at line I-I of FIG. 1.

The user applies the earplug 2 by first compressing the sound attenuating element 4 to temporarily reduce the diameter D. This compression may be achieved by the user rolling the sound attenuating element 4 between their hands and/or fingers. The user then inserts the first end 6 of the reduced diameter earplug 2 into the ear canal. The full recovery resilient material of the earplug 2 then expands to obstruct the ear canal and thus provides sound attenuation. When the earplug 2 is in the inserted position, the second end 8 remains at the opening of the ear canal or extends therefrom.

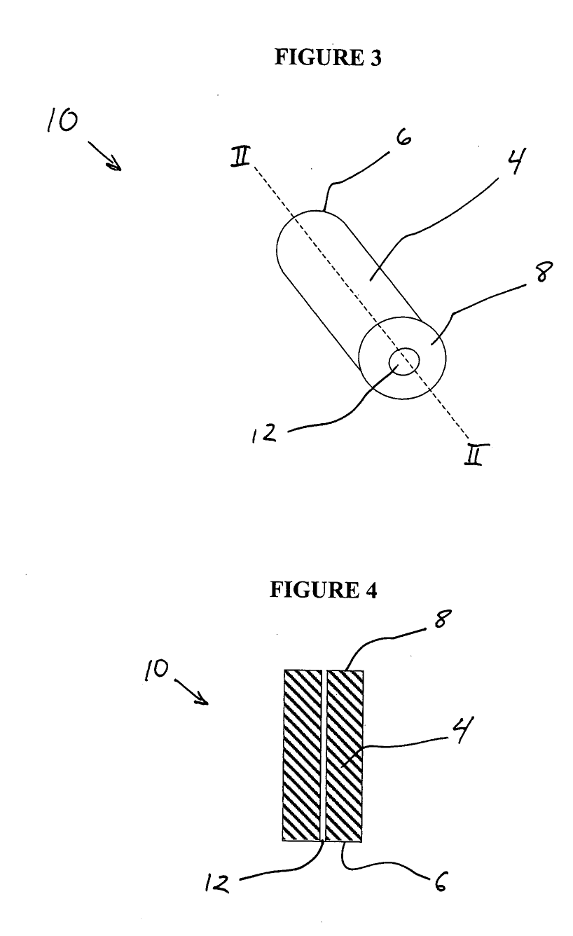

FIG. 3 shows an earplug 10 according to another embodiment of the invention. The earplug 10 includes the sound attenuating element 4 having ends 6 and 8, as shown in FIG. 1, and further includes a hole 12 formed therein.

FIG. 4 shows a cross-section of the earplug 10 taken along line II-II of FIG. 3. As shown, the hole 12 extends longitudinally along a central axis of the sound attenuating element 4 and opens to an exterior of the earplug 10 at the first and second ends 6 and 8. The hole 12 is substantially cylindrical in cross-section.

The hole 12, as shown and described, may be used to receive and retain items within the sound attenuating element 4 of the earplug 10.

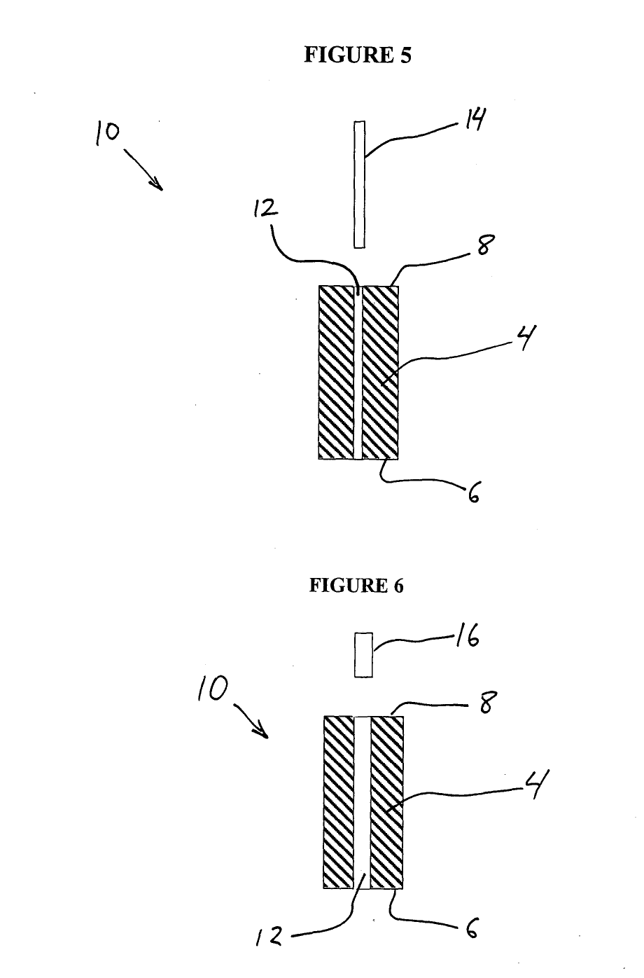

For example, as shown in FIG. 5, the hole 12 may receive and retain a stem 14. The stem 14 is a rigid or semi-rigid cylindrical element which generally corresponds in shape and diameter to the hole 12. The stem 14 is inserted in the hole 12 and bonded therein to the sound attenuating element 4 with a bonding agent, for example, a glue. The stem 14 includes a longitudinal length greater than, equal to, or less than that of the hole 12, thus allowing the stem embed within the element 4 or extend therefrom, as desired.

When inserted and retained within the sound attenuating element 4, the stem 14 provides the earplug 10 with a degree of rigidity which facilitates insertion of the earplug 10 into the ear canal of the wearer.

The hole 12 may further be utilized to receive and retain a detectable insert 16, as shown in FIG. 6. The detectable insert 16 is composed of any readily detectable material, such as, for example a material which is metal, magnetic, or x-ray detectable.

Still further, the hole 12 may be used to allow a certain level of sound to pass through the earplug 10, thus providing a prescribed reduction in the attenuation provided by the plug. Alternatively, the hole 12 of FIG. 4 may receive and retain communication equipment, such as a transmitter or receiver, for facilitating communication with the wearer of the earplug 10.

In another example, the hole 12, as shown in any of FIGS. 4-6, may be used to receive and retain an end of a cord to attach the earplug 10 with another earplug.

Of course the invention contemplates additional configurations, contemplations, and uses of the hole 12 formed in the sound attenuating element 4 of the earplug 10.

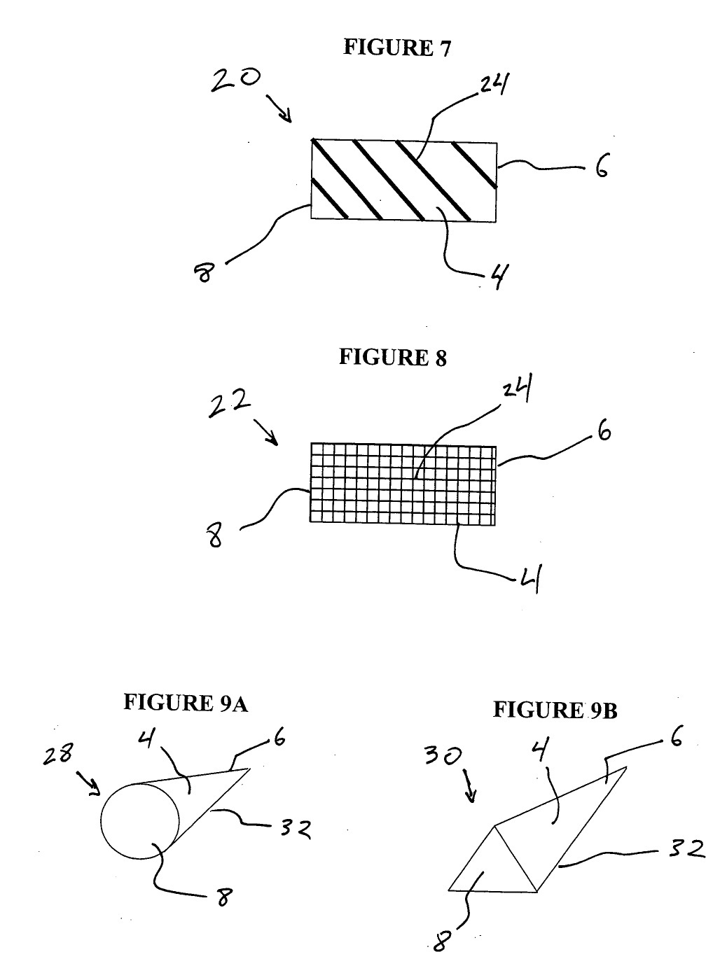

FIGS. 7 and 8 show earplugs 20 and 22, respectively, in another embodiment of the invention. The earplugs 20 and 22 include the sound attenuating element 4 having first and second ends 6 and 8, as shown and described with reference to the earplug 2 of FIG. 1. The earplugs 20 and 22 further include scoring 24 formed on an exterior surface thereof. The scoring 24 comprises, for example, a pattern forming any type of ornamental design and is shown here, representatively, as helical lines (FIG. 7) and intersecting perpendicular lines (FIG. 8). The scoring 24 may be formed on the surface of the earplugs 20 and 22, respectively, as inset and/or protruding features. That is, the scoring 24 may have an inscribed or raised appearance.

FIGS. 9A and 9B show earplugs 28 and 30 in additional embodiments of the invention. Therein, the earplugs 28 and 30 each include the sound attenuating element 4 having first and second ends 6 and 8, as shown and described with reference to the earplug 2 of FIG. 1. The earplugs 28 and 30 further include angled shaping 32 formed in sides of the sound attenuating element 4 along a longitudinal direction of the earplug 30. That is, essentially, portions of the generally cylindrical sound attenuating element 4 are removed to form the angled shaping 32. Such shaping 32 is shown in FIGS. 9A and 9B as forming rounded and planar side portions, respectively, of the sound attenuating element 4. Such side portions, due to their angled nature, taper to resultantly form the conical and pyramidal shaped earplugs 28 and 30 as shown in the drawings. Of course, such features are purely exemplary and the angled shaping 32 could take any advantageous form in order to shape the resulting earplug 30 as desired.

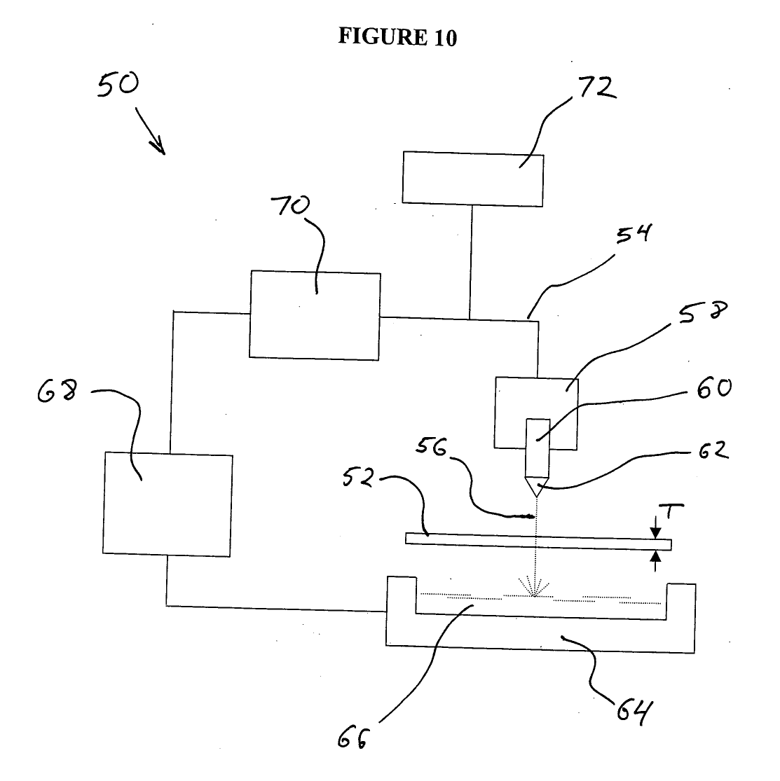

A method 50 of manufacturing the earplug of the invention, as shown and described herein, is now provided with reference to FIGS. 1-10.

As mentioned, the sound attenuating element 4 is composed of a full recovery, compressible, resilient material. Firstly, this material is produced in a form of a sheet 52. The material sheet 52 may be dimensioned as desired and preferably includes a thickness T generally equivalent to the length L of the sound attenuating element 4. The sheet 52 is produced according to any viable method. For example, the resilient material sheet 52 may be formed in a casting process where the material is deposited on a substrate and allowed to cure thereon with or without application of heat, chemical treatment, etc.

The sheet 52 has a width and a length nearly equal to an even multiple of the diameter D of the sound attenuating element 4. For example, if D=Xcm then the width of the sheet 52 may equal approximately 10 Xcm, or slightly more, and the length may equal approximately 50 Xcm, or slightly more. In this way, when the sound attenuating elements 4 are formed from the sheet 52, as discussed herein, waste is kept to a minimum.

Once produced, the sheet 52 is brought proximate a water jet assembly 54. For example, the material sheet 52 is transported to the water jet assembly 54 by a conveying device such as a conveyor belt. Alternatively, the water jet assembly 54 may be translatable and may be positioned proximate the stationary or semi-stationary material sheet 52.

Once the sheet 52 of the sound attenuating resilient material and the water jet assembly 54 are proximate one another, the assembly 54 is activated so as to emit a high pressure stream of water 56. The stream of water 56 is used to cut individual sound attenuating elements 4 from the sheet 52. That is, the high pressure water stream 56 severs the resilient material as desired to form the sound attenuating elements 4.

The water jet assembly 54 includes a cutting head 58 having a nozzle 60 disposed thereon. The nozzle includes a channel formed therein for allowing passage of the high pressure water stream 56. A jewel 62 is disposed on the nozzle 60. The jewel includes an orifice which is in fluid communication with the channel to permit passage of the water stream 56 from the nozzle 60, through the jewel 62, to the material sheet 52.

The jewel 62 is composed of a suitable material with a hardness sufficient to maintain precise dimensions of the orifice despite potentially degrading forces of the high pressure water stream 56. For example, in a preferred embodiment, the jewel 62 is composed of a ruby, sapphire, or diamond.

The water jet assembly 54 further includes a catch tank 64 preferably disposed beneath the cutting head 58 and proximate the material sheet 52. The catch tank 64 is utilized to catch and retain water and sediment from the water jet cutting process, herein indicated at reference numeral 66. In one embodiment a slat is disposed atop the catch tank 64 for supporting the material sheet during application of the water jet.

The catch tank 64 is further configured to direct the spent water and sediment particles 66 to a filtration system 68. The filtration system 68 filters the water/sediment mixture 66 thus reclaiming the water and directing the same for re-use in the water jet process.

The water jet assembly 54 further includes a pump 70 for generating the required high pressure to form the water stream 56. The pump 70 generally comprises any means suitable for attaining a desired water pressure for the water stream 56. For example, the pump 70 may be a 10-50 Hp pump, preferably, a direct drive crankshaft style pump or an intensifier style pump. In any event, the pump 70 is sufficient to provide a water pressure in the water stream 56 at the orifice of the jewel 62 of approximately 30,000-100,000 psi and preferably 50,000 psi.

The water jet assembly 54 further includes a controller 72 which monitors and administers the functioning of the water jet assembly 54. For example, the controller 72 controls operation of the pump 70 and hence pressurization of the water stream 56, movement of the cutting head 58 relative the material sheet 52, and/or movement of the material sheet 52 relative the cutting head 58, and filtration of the spent water/sediment mixture 66. That is, the controller 72 monitors and regulates all properties of the water jet cutting operation including, but not limited to, cutting speed, cutting depth, kerf width, flow rate, cutting quality (i.e., surface finish), piercing (i.e., stationary, dynamic, wiggle, etc.), scribing, machineability (i.e., cutting index), jet lag, striation marks, and taper. The controller 72 preferably comprises a computer and a software package such as, for example, the software package commercially available as OMAX.

In one embodiment, the controller 72 regulates and controls movement of the cutting head 58 in at least two directions and up to five directions to facilitate precise application of the high pressure water stream.

As addressed above, the high pressure water stream 56 produced by the water jet assembly 54 is used to sever individual sound attenuating elements 4 from the sheet 52 of the compressible, resilient material. The sound attenuating element 4 of earplug 2 as shown in FIG. 1, which includes a substantially cylindrical shape and length L generally equivalent to the thickness T of the material sheet 52, is separated from the sheet 52 by applying the high pressured water stream 56 to the sheet 52. Particularly, a side edge of the water stream 56 engages an edge of the material sheet 52 in a substantially perpendicular manner and essentially cuts the compressible, resilient material along the circumference of the resulting cylindrical sound attenuating element 4. In this way, the water stream 56 of the water jet assembly 54 cuts entirely through the material sheet 52 about the circumference of one of the ends 6,8 of the resulting sound attenuating element 4. In cutting perpendicularly through the entire thickness T of the material sheet 52, as such, the earplug 2 with length L and diameter D is realized. Alternatively, the desired perpendicular circumferential cutting may be achieved by simply piercing a top surface of the material sheet 52 by activating the water jet there atop at a desired location.

The earplug 10 as shown in FIGS. 3-6 is formed utilizing the water jet assembly 54 as follows. First, the sheet 52 is formed of the full recovery, resilient, compressible material of which the pertinent sound attenuating element 4 is composed. As mentioned above, the material sheet 52 is formed by any suitable process, preferably by casting. Then, the sound attenuating element 4 is separated from the material sheet 52 as described above concerning the earplug 2 with reference to FIGS. 1, 2, and 10. That is, the high pressured stream 56 produced by the water jet assembly 54 perpendicularly contacts the material sheet 52 to sever the sound attenuating element 4 therefrom.

Alternatively, of course, the sound attenuating element 4 may first be produced by conventional means such as molding, extrusion, die cutting, etc., and then subsequently subject to the water jet cutting procedure of the invention in order to form the hole 12, as described below.

Once the sound attenuating element 4 of the earplug 10 is produced, the hole 12 is bore therein. Particularly, the individual sound attenuating element 4 is positioned beneath the cutting head 58 of the water jet assembly 54, proximate the jewel 62. At this stage, the controller 72 refrains the water jet assembly 54 from producing the high pressure water stream 52. Once the sound attenuating element 4 is properly positioned beneath the cutting head 58, the water jet assembly 54 is activated so as to produce the water stream 52 which pierces the compressible, resilient material forming the element 4 to bore the hole 12 therein. Any suitable piercing technique may be employed to form the desired hole 12 including, but not limited to, wiggle, dynamic, stationary, and low pressure piercing.

As desired, the pressure of the water stream 56, the width of the stream 56 as emitted from the orifice of the jewel 62, and the time of exposure of the stream 56 to the element 4 may be adjusted to produce the desired hole 12. Once the hole 12 is formed, the earplug 10 may then be further processed as desired to include various inserted items such as the stem 14 and the detectable insert 16.

Alternatively, the earplug 10 of FIGS. 3-6 may be manufactured by first forming the hole 12 in the material sheet 52 through piercing as described above and then severing the sound attenuating element 4 therefrom. That is, the high pressure water stream 56 may first be applied to the sheet 52 in order to pierce the desired hole 12. Then, the stream may be re-applied to cut the sound attenuating element 4 from the sheet 52, thus forming the earplug 10. Particularly, the water stream 52 may pierce the hole 12 in the material sheet 52 substantially perpendicularly thereto and then the stream 56 may cut along the circumference of one of the ends 6,8 of the sound attenuating element, around the bored hole 12, to thus sever the earplug 10 from the material sheet 52.

As discussed, the earplugs 20 and 22 include the scoring 24. See, FIGS. 7-8. The earplugs 20 and 22 are formed by first producing the sound attenuating element 4. Such element 4 may be formed by the water jet cutting application as described herein or by conventional methods such as extrusion, molding, die cutting, etc.

Once formed, the sound attenuating element 4 is brought proximate the water jet cutting assembly and, particularly, substantially beneath the jewel 62 of the cutting head 58. When the sound attenuating element 4 is properly positioned, the controller 72 activates the water jet assembly 54 such that the high pressure water stream 56 is emitted from the orifice of the jewel 62. The edge of the stream 56 is brought into contact with sides of the sound attenuating element 4 to essentially ablate a portion of the compressible, resilient material composing the element, thus forming the scoring 24. That is, the scoring 24 is etched into an outer surface of the sound attenuating element 4 to form the ornamental patterns as shown on earplugs 20 and 22.

The pressure of the water stream 56, the width of the stream 56 as emitted from the orifice of the jewel 62, and the time of exposure of the stream 56 to the element 4 may be adjusted as desired to produce the scoring 24, as desired, having a specific width and depth. The sound attenuating elements 4 of earplugs 20 and 22 may further be rotated beneath the jewel 62 such that the scoring 24 extends around the circumference of the elements 4 in directions, for example, perpendicular to or helical with respect to a longitudinal axis of the earplugs 20 and 22. Alternatively, the cutting head 58 may be disposed so as to rotatably move about the sound attenuating elements 4 to form the scoring 24 as shown in the drawings. Alternatively, it is understood that said scoring 24 may be formed on only a select portion or portions of the elements 4, rather than across the entire surface area as is shown for exemplary purposes in the drawings.

Alternatively, the outer surface of the sound attenuating element 4 may be selectively removed through said scoring 24 to form desired features protruding from the surface. That is, the high pressure water stream 56 may be used to ablate outer surface areas of the element 4 relative to other remaining portions which are left to protrude and thus form a desired pattern, design, etc.

FIGS. 9A and 9B show earplugs 28 and 30, respectively, including the sound attenuating element 4 having angled shaping 32 formed in sides thereof. That is, portions of the generally cylindrical sound attenuating element 4 are removed to form the angled shaping 32 which, consequently, results in the conical and pyramidal shaped earplugs 28 and 30, as shown.

The earplugs 28 and 30 are manufactured by first forming the sound attenuating elements 4 by suitable means including the water jet cutting process described herein or by conventional formation means such as molding and extrusion. Next, the sound attenuating elements 4 are brought proximate the waterjet assembly 54 substantially beneath the cutting head 58 and the jewel 62. The assembly 54 is activated so as to emit the high pressure water stream 56. The edge of the stream 56 is brought into contact with or, alternatively, the stream 56 pierces the compressible, resilient material composing the sound attenuating element 4 and essentially ablates the same to form the angled shaping 32. The angled shaping 32, as shown in FIGS. 9A and 9B, comprises curved side portions and side portions of the sound attenuating element 4, both of which taper to end 6, but may also include any other desired configuration.

Alternatively, the earplugs 28 and 30 may be formed in a singe step water jet process, according to the invention. Particularly, the resilient material sheet 52 is formed as discussed herein and positioned proximate or beneath the water jet cutting assembly 54. The assembly 54 is activated so as to emit the water stream 56 at a desired angle relative to the resilient material sheet 52. The angled water stream 56 is brought into contact with the material sheet 52 so as to cut the sheet 52 at the angle. The water jet assembly 54 then operates to traverse the water stream 56 across the material sheet 52 in order to sever therefrom the earplugs 28 and 30 in a single cutting process.

For example, where the earplug 28 having a conical shape is desired, the angled water stream 56 is brought into contact with the sheet 52 and then traversed across the sheet so as to trace the circumference of the end 8. By maintaining the angle of the water stream 56 relative to the longitudinal axis of the particular sound attenuating element 4, the earplug 28 is resultantly severed from the material sheet 52 in a single cut process.

Similarly, the earplug 30 may be formed in a single step water jet cutting process by engaging the angled water stream 56 with the resilient material sheet, as discussed above, and then tracing the perimeter of the end 8 while maintaining the angle relative to the longitudinal axis of the earplug 30.

The water jet cutting assembly 54 as described and discussed herein enables the sheet material 52 to be cut at a straight line rate of approximately 1000 linear inches per minute. In a preferred embodiment, when forming the sound attenuating element 4, the material sheet 52 is cut at approximately 100 linear inches per minute.

A single cutting head 58 has been shown and described herein for exemplary purposes only. The water jet assembly 54 may further include additional cutting heads and additional corresponding jewels to produce multiple high pressure water streams. Such multiple streams are used to simultaneously cut multiple sound attenuating elements from the material sheet. Alternatively, the multiple high pressure streams are utilized to sever and further process the sound attenuating elements. For example, a first cutting head may form the hole 12 in the material sheet 52 while a second cutting head nearly simultaneously severs the resulting holed sound attenuating element 4 from the material sheet 52. Further, the first cutting head can cut the sound attenuating element from the material sheet while the second cutting head then ablates portions thereof to from the angled shaping to produce the earplugs 20 or 22. Still, further the multiple cutting heads may simultaneously work to cut a single sound attenuating element from the material sheet, thus allowing the element to include a complex three-dimensional configuration.

The earplugs and sound attenuating elements of the generally cylindrical shape are discussed herein for exemplary, non-limiting purposes. The invention contemplates earplugs and sound attenuating elements of various shapes and configurations. For example, the water jet assembly 54 may be used to cut ellipsoidal, spherical, or polygonal shapes or any combinations thereof.

Earplugs composed of a non-monolithic material, i.e., composed of a layered material, may be manufactured by the method of the invention by first forming a layered laminate of the desired earplug materials. For example, different density foams may be formed together or bonded together in layers to form a layered material sheet. The desired earplugs or sound attenuating elements may then be severed from the sheet by the water jet assembly 54, as discussed above.

The water jet formation of earplugs, discussed herein, efficiently and precisely produces compressible earplugs of exacting measurements and quality. The kerf width of the waterjet is extremely small, thus very little compressible, resilient material is wasted. That is, more earplugs may be extracted from the material sheet by the method described herein than may be extracted by conventional methods such as die-cutting. Essentially no heat is evolved in the water jet cutting process, thus the resulting earplugs are not thermally degraded. Further, the water jet cuts cleanly through the resilient, compressible material without compressing the same. Thus, no permanent deformation or pinching of the produced earplugs result from the method of the invention. Finally, due to the precision cutting available by the water jet assembly and due to the precision of the controller, the earplugs of the invention are produced rapidly, accurately, and consistently.

While preferred embodiments have been shown and described, various modifications and substitutions may be made thereto without departing from the spirit and scope of the invention. Accordingly, it is to be understood that the present invention has been described by way of illustrations and not limitation.

1. A method of forming an earplug, comprising:
providing a sheet of a compressible, resilient material;
positioning the sheet proximate a water jet assembly;
activating the water jet assembly to emit a high pressure water stream; and
contacting the sheet with the water stream cutting the sheet and severing the earplug from the sheet.

2. The method of claim 1, wherein said providing the sheet comprises forming the sheet to include a thickness substantially equal to a length of the earplug.

3. The method of claim 1, wherein said cutting the sheet comprises contacting the sheet with the water stream substantially perpendicular to a top surface thereof and traversing circular patterns on the sheet to sever the earplug from the sheet, the earplug being substantially cylindrical in shape.

4. The method of claim 1, wherein said positioning the sheet comprises conveying the sheet using a conveyor belt and depositing the sheet on a slat.

5. The method of claim 1, wherein said activating the water jet assembly comprises a computer controller controlling a pump for generating the high pressure water stream.

6. The method of claim 5, further comprising catching the high pressure water stream in a catching tank after said cutting and severing, filtering said water after said catching, and pressurizing said water after said filtering.

7. The method of claim 1, wherein the high pressure water stream includes a pressure of approximately 50,000 pounds per square inch.

8. The method of claim 1, wherein the high pressure water stream is emitted through an orifice having a diameter of approximately 0.005 to 0.010 inches.

9. The method of claim 8, wherein said orifice is formed in a ruby or a sapphire or a diamond jewel.

10. The method of claim 1, wherein said cutting comprises forming a kerf in the sheet, the kerf having a width of approximately 0.005 to 0.020 inches.

11. The method of claim 1, further comprising piercing the sheet with the high pressure water stream forming a hole therein then said cutting the sheet around the hole such that the severed earplug includes the hole.

12. The method of claim 1, further comprising piercing the severed earplug with the high pressure water stream forming a hole therein, said piercing occurring after said severing.

13. The method of claim 12, wherein the hole is formed extending along a longitudinal axis of the earplug entirely through the earplug.

14. The method of claim 12, further comprising inserting an item in the hole and bonding the item to the earplug at the hole.

15. The method of claim 14, wherein the item comprises at least one of a stem, a metal detectable insert, or an end of a cord.

16. The method of claim 1, further comprising contacting a portion of a surface of the earplug with the high pressure water stream to ablate the portion, forming a detail on the surface.

17. The method of claim 16, wherein the detail is etched into the surface so as to be inset therein.

18. The method of claim 16, wherein the portion of the surface is removed to form the detail in relief.

19. The method of claim 16, wherein the detail comprises at least one of an a pattern and an angled shaping.

20. The method of claim 19, wherein the angled shaping comprises tapered side portions providing the earplug with at least one of a conical, frustoconical, an pyramidal shape.

21. The method of claim 1, wherein said contacting the sheet comprises engaging the sheet with the water stream at an angle to a longitudinal axis of the earplug and wherein said cutting the sheet and said severing the earplug comprises tracing an end of the earplug on a top surface of the sheet while maintaining the angle of the water stream relative to the longitudinal axis to form the earplug including a conical or pyramidal shape.

22. A method of forming an earplug, comprising:
forming a sheet of resilient, compressible full-recovery foam material;
conveying the foam sheet to a water jet cutting assembly;
depositing the foam sheet on a slat;
pressurizing water in the water jet cutting assembly with a pump;
delivering the pressurized water through a jewel in a high pressure stream;
contacting the foam sheet with the high pressure stream; and
maneuvering the high pressure stream to cut the foam sheet, shape the earplug, and sever the earplug from the foam sheet.

23. The method of claim 22, wherein said contacting the sheet comprises engaging the sheet with the water stream at an angle to a longitudinal axis of the earplug and wherein said cutting the sheet and said severing the earplug comprises tracing an end of the earplug on a top surface of the sheet while maintaining the angle of the water stream relative to the longitudinal axis to form the earplug including a conical or pyramidal shape.

24. An earplug, comprising:
a portion formed by water jet cutting.

25. The earplug of claim 24, wherein said portion comprises at least one of a shape of the earplug, an ornamental detail of the earplug, and a hole formed in or through the earplug.

26. The earplug of claim 24, wherein said portion formed by water jet cutting comprises a cylindrical, conical, or pyramidal outer surface of the earplug.

27. The earplug of claim 26, wherein said portion formed by water jet cutting further comprises a hole formed through the earplug along a longitudinal axis thereof.

28. The earplug of claim 27, further comprising at least one of a stem, a metal detectable insert, or an end of a cord disposed in the hole.

29. The earplug of claim 24, further comprising a foam body portion, wherein the portion formed by water jet cutting comprises at least one of an outer surface of the body portion, an ornamental detail at the outer surface, and hole formed through the body portion along a longitudinal axis thereof.
